(19) United States
(12) Patent Application Publication
(10) Pub. No.: US 2001/0051168 A1
(43) Pub. Date: Dec. 13, 2001

(54) MAKEUP KIT COMBINING FILM-FORMING COMPOSITIONS

(76) Inventors: ROLAND RAMIN, PARIS (FR); INGRID BRENNE, L'HAY-LES-ROSES (FR)

Correspondence Address: FINNEGAN HENDERSON FARABOW, GARRETT & DUNNER LLP, 1300 I STREET NW, WASHINGTON, DC, 200053315

(21) Appl. No.: 09/408,120

(22) Filed: Sep. 29, 1999

(30) Foreign Application Priority Data
Sep. 30, 1998 (FR) 98 12234

(51) Int. Cl. A61K 7/04; A61K 7/021; A61K 6/00; A61K 7/00; A01N 25/00; A61K 47/00

(52) U.S. Cl. 424/401; 424/61; 424/63; 514/772; 514/781

(57) ABSTRACT

A makeup kit comprising a first film-forming composition capable of forming a first film having a hardness d1 ranging from 20 to 85 seconds, and a second film-forming composition capable of forming, on the first film, a second film having a hardness d2 such that d2−d1 is greater than or equal to 10 seconds. This kit is intended in particular for making up keratinous substances. Also, a method for making up keratinous substances.

The present invention relates to a makeup kit intended for a new type of makeup, combining two film-forming compositions which can be applied to the skin both of the face and of the body, to the lips and to the exoskeletal appendages such as the nails, eyelashes, eyebrows or hair. The invention also relates to a bilayer makeup method.

Each composition can be a nail varnish, a rouge or eyeshadow, a foundation, a lip makeup product or a body makeup product.

The nail varnish compositions generally comprise a film-forming polymer either dissolved in an organic solvent or dispersed in particle form in an aqueous medium. Such varnishes are described, for example, in the documents U.S. Pat. No. 4,158,053 and FR-A-2578741, the disclosures of each which are incorporated by reference herein.

These nail varnishes, following application to the nail of one or more layers of the composition, and drying, generally lead to the formation of a smooth, continuous and homogeneous film which is glossy or matte. Certain films also can show good cosmetic properties, such as a good hold and, in particular, good adhesion to the nail, and good water resistance, frictional resistance and flaking resistance.

With the progress of fashion, consumers, who are more demanding, are looking for new makeup products which allow original or special makeup effects to be obtained. There is therefore a need to provide a makeup product whose application to a substrate such as the skin or the exoskeletal appendages of human beings makes it possible to obtain a makeup effect which is different from that of the continuous, homogeneous films currently obtained with the products available on the market.

The object of the present invention is therefore to provide a makeup composition which makes it possible to obtain a non-homogeneous makeup and/or a discontinuous film on the skin and on the exoskeletal appendages whilst exhibiting a good hold over time.

The inventors have noted that a novel type of makeup for the skin and/or exoskeletal appendages can be obtained by means of the application of two specific film-forming compositions. These compositions make it possible to obtain a makeup featuring small fissures or cracks distributed more or less regularly over the surface; the makeup obtained therefore can exhibit an original crackle effect. Furthermore, the makeup can exhibit good adhesion to the made-up substrate and a good hold, in particular with respect to friction and flaking.

More specifically, the invention provides a makeup kit comprising a first composition comprising a cosmetically acceptable medium and at least one first film-forming polymer, the first composition being capable of forming a first film having a hardness d1 ranging from 20 to 85 seconds, and a second composition comprising a cosmetically acceptable medium and at least one second film-forming polymer, the second composition being capable of forming a second film having a hardness d2 such that d2−d1 is greater than or equal to 10 seconds.

The invention additionally provides a method of making up the skin, lips and/or exoskeletal appendages of a human being, which comprises applying to the skin, lips and/or exoskeletal appendages a first layer of a first film-forming composition comprising a cosmetically acceptable medium and at least one first film-forming polymer, the first composition being capable of forming a first film having a hardness d1 ranging from 20 to 85 seconds, then in applying to at least one part of the first layer a second layer of a second film-forming composition comprising a cosmetically acceptable medium and at least one second film-forming polymer, the second composition being capable of forming a second film having a hardness d2 such that d2−d1 is greater than or equal to 10 seconds.

Advantageously, the two compositions according to the invention are packaged in separate containers or compartments, and are accompanied by appropriate means of application, identical or different, such as coarse or fine brushes, pens and sponges.

According to the invention, the second layer is applied to at least one part of the first layer, or even the entirety of the first layer, following drying of the first layer. It can be applied either to one of the ends of the first layer, or in the middle, or else discontinuously, in particular in the form of geometric, symmetric or asymmetric motifs (for example, in the form of dots, squares, circles or stars) which are in random or ordered distribution and have sharp or fuzzy outlines.

This brayer architecture can be adapted for all makeup products for the skin both of the face and of the scalp and body, the mucosae, such as the lips and the inside of the lower eyelids, and the exoskeletal appendages, such as the nails. The second layer can be applied with any type of instrument (sponge, finger, coarse or fine brush, pen, etc.). This architecture may also be applied to makeup accessories such as false nails, false eyelashes, hairpieces, or else discs or patches which adhere to the skin or lips (of the beauty-spot type).

The invention additionally relates to a made-up substrate whose makeup is capable of being obtained by the method according to the invention.

The invention additionally provides for the use of the first and/or second compositions as defined above to obtain a crackle-surface makeup having good hold.

The inventors have discovered that the application of two compositions differing in film hardness, according to the invention, makes it possible to obtain a crackle-surface makeup which exhibits good adhesion to the made-up substrate and a good hold with respect in particular to friction and to flaking. Moreover, the application of the second composition directly to the unmade-up substrate leads to the formation of a crackle-surface film; however, the film is very crumbly and therefore has a very poor hold with respect to friction. The application of the first composition according to the invention therefore makes it possible to obtain a crackle-surface makeup having a good hold with respect to friction.

The hardness of the film is measured for a layer 300 μm thick (before drying) deposited on a glass plate heated at 30° C. and after drying for 23 hours in the ambient atmosphere and then for 1 hour at 70% relative humidity for a solvent-based composition or at 50% relative humidity for a water-based composition. The hardness of the film obtained is measured in accordance with the standard ASTM D4366-95 or the standard NF-T 30-016 (December 1991), with the aid of a Persoz pendulum, the disclosures of each which are incorporated by reference herein.

For the implementation of the method according to the invention, the hardness d2 must be such that d2−d1 ranges from 10 to 360 seconds and, preferably, from 10 to 120 seconds.

Preferably, d1 can range from 20 to 85 seconds and d2 can range from 30 to 180 seconds. More preferably, d1 can range from 20 to 40 seconds and d2 can range from 80 to 120 seconds.

In accordance with the method of the invention, the first and second compositions can comprise, independently of one another, an aqueous medium or an organic solvent medium.

Therefore, in accordance with a first embodiment of the invention, the first and second compositions can comprise an identical or different aqueous medium.

In accordance with a second embodiment of the invention, the first composition can comprise an aqueous medium and the second composition can comprise an organic solvent medium.

In accordance with a third embodiment of the invention, the first composition can comprise an organic solvent medium and the second composition can comprise an aqueous medium.

In accordance with a fourth embodiment of the invention, the first and second compositions can comprise an identical or different organic solvent medium.

In order to promote the formation of the cracks or fissures, which in particular are distributed regularly over the surface of the second film, it is possible to employ means for accelerating the drying of the second layer following the application of the second film-forming composition. These means can be, for example, a source of heat (hairdryer, halogen lamp) or of pulsed air (fan).

Among the film-forming polymers which can be used in the composition of the present invention, mention may be made of synthetic polymers, of free-radical type or polycondensate type, polymers of natural origin, and mixtures thereof.

By free-radical film-forming polymer is meant a polymer obtained by polymerization of monomers containing unsaturation, especially ethylenic unsaturation, each monomer being capable of undergoing homopolymerization (in contradistinction to polycondensates).

The film-forming polymers of free-radical type can in particular be vinyl polymers or copolymers, especially acrylic polymers.

The film-forming vinyl polymers can result from the polymerization of monomers containing ethylenic unsaturation and having at least one acid group, and/or esters of these acidic monomers, and/or amides of these acidic monomers.

Preference is given to the use of anionic free-radical film-forming polymers, i.e. polymers comprising at least one monomer having an acid group.

As acid group-bearing monomer, it is possible to use α,β-ethylenic unsaturated carboxylic acids such as acrylic acid, methacrylic acid, crotonic acid, maleic acid and itaconic acid. Preference is given to the use of (meth)acrylic acid and crotonic acid, and more preferably (meth)acrylic acid.

The esters of acidic monomers are selected advantageously from esters of (meth)acrylic acid (also called (meth)acrylates), especially alkyl (meth)acrylates, in particular C₁-C₂₀ alkyl (meth)acrylates, preferably C₁-C₈ alkyl (meth)acrylates, aryl (meth)acrylates, in particular C₆-C₁₀ aryl (meth)acrylates, and hydroxyalkyl (meth)acrylates, in particular C₂-C₆ hydroxyalkyl (meth)acrylates. Among the alkyl (meth)acrylates, mention may be made of methyl methacrylate, ethyl methacrylate, butyl methacrylate, isobutyl methacrylate, 2-ethylhexyl methacrylate and lauryl methacrylate.

Among the hydroxyalkyl (meth)acrylates, mention may be made of hydroxyethyl acrylate, 2-hydroxypropyl acrylate, hydroxyethyl methacrylate and 2-hydroxypropyl methacrylate.

Among the aryl (meth)acrylates, mention may be made of benzyl acrylate and phenyl acrylate.

The particularly preferred esters of (meth)acrylic acid are the alkyl (meth)acrylates.

In accordance with the present invention, the alkyl group of the esters can be either fluorinated or perfluorinated; in other words, some or all of the hydrogen atoms of the alkyl group are substituted by fluorine atoms.

As amides of acidic monomers, mention may be made, for example, of the (meth)acrylamides, and especially the N-alkyl(meth)acrylamides, in particular those comprising C₂-C₁₂ alkyl. Among the N-alkyl(meth)-acrylamides, mention may be made of N-ethylacrylamide, N-t-butylacrylamide and N-t-octylacrylamide.

The film-forming vinyl polymers may also result from the homopolymerization or copolymerization of at least one monomer selected from vinyl esters and styrenic monomers. In particular, these monomers can be polymerized with acidic monomers and/or their esters and/or their amides, such as those mentioned above. As examples of vinyl esters, mention may be made of vinyl acetate, vinyl neodecanoate, vinyl pivalate, vinyl benzoate and vinyl t-butylbenzoate. As styrenic monomers, mention may be made of styrene and alpha-methylstyrene.

The list of monomers given is not limitative, and it is possible to use any monomer known to the person skilled in the art which falls within the categories of acrylic and vinyl monomers (including the monomers modified by a silicon-containing chain).

As film-forming acrylic polymers in aqueous dispersion which can be used in accordance with the invention, mention may be made of those sold under the names NEOCRYL XK-90®, NEOCRYL A-1070®, NEOCRYL A-1090®, NEOCRYL BT-62®, NEOCRYL A-1079®, NEOCRYL A-523® by the company ZENECA and DOW LATEX 432® by the company DOW CHEMICAL.

Among the polycondensates which can be used as film-forming polymer, mention may therefore be made of anionic, cationic, nonionic or amphoteric polyurethanes, acrylic polyurethanes, polyvinylpyrrolidone polyurethanes, polyester polyurethanes, polyether polyurethanes, polyureas, polyurea/polyurethanes, and mixtures thereof.

The film-forming polyurethane can be, for example, a polyurethane copolymer, a polyurea/urethane copolymer or an aliphatic, cycloaliphatic or aromatic polyurea copolymer comprising, alone or in a mixture:

at least one sequence originating from an aliphatic and/or cycloaliphatic and/or aromatic polyester, and/or

at least one silicon-containing sequence, branched or otherwise, for example polydimethylsiloxane or polymethylphenylsiloxane, and/or

at least one sequence comprising fluorinated groups.

As film-forming polyurethane polymers in aqueous dispersion which can be used in accordance with the invention, mention may be made in particular of the polyester polyurethanes sold under the names AVALURE UR-405®, AVALURE UR-410®, AVALURE UR-425® SANCURE 2060® by the company GOODRICH and the polyether polyurethanes sold under the names SANCURE 878® by the company GOODRICH, and NEOREX R 970® by the company ICI.

Among the film-forming polycondensates, mention may also be made of polyesters, polyester amides, fatty-chain polyesters, polyamides and epoxy ester resins, resins resulting from the condensation of formaldehyde with an arylsulphonamide, and arylsulphonamide epoxy resins.

The polyesters can be obtained, in a known manner, by polycondensation of dicarboxylic acids with polyols, especially diols.

The dicarboxylic acid can be aliphatic, alicyclic or aromatic. Examples of such acids which may be mentioned are oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid, and 2,6-naphthalenedicarboxylic acid. These dicarboxylic acid monomers can be used alone or in a combination of at least two dicarboxylic acid monomers. Among these monomers, it is preferred to select phthalic acid, isophthalic acid and/or terephthalic acid.

The diol can be selected from aliphatic, alicyclic and aromatic diols. Preference is given to the use of a diol selected from ethylene glycol, diethylene glycol, triethylene glycol, 1,3-propanediol, cyclohexane dimethanol and 1,4-butanediol. Other polyols which can be used include glycerol, pentaerythritol, sorbitol and trimethylol propane.

The polyester amides can be obtained in a manner similar to the polyesters, by polycondensation of diacids with diamines or amino alcohols. As the diamine, it is possible to use ethylenediamine, hexamethylenediamine or meta- or paraphenylenediamine. As the amino alcohol, it is possible to use monoethanolamine.

The polyester may additionally comprise at least one monomer which bears at least one group —SO₃M in which M represents a hydrogen atom, an ammonium ion NH₄⁺ or a metal ion, such as, for example, an Na⁺, Li⁺, K⁺, Mg²⁺, Ca²⁺, Cu²⁺, Fe²⁺ or Fe³⁺ ion. It is also possible to use, in particular, a bifunctional aromatic monomer comprising such a group —SO₃M.

The aromatic nucleus of the bifunctional aromatic monomer additionally bearing a group —SO₃M, as described above, can be selected, for example, from the benzene, naphthalene, anthracene, diphenyl, oxydiphenyl, sulphonyldiphenyl and methylenediphenyl nuclei. As examples of a bifunctional aromatic monomer additionally bearing a group —SO₃M, mention may be made of sulphoisophthalic acid, sulphoterephthalic acid, sulphophthalic acid and 4-sulpho-2,7-naphthalenedicarboxylic acid.

In the compositions which are the subject of the invention, preference is given to the use of isophthalate/sulphoisophthalate-based copolymers, and more particularly to copolymers obtained by condensation of diethylene glycol, cyclohexane dimethanol, isophthalic acid, sulphoisophthalic acid. Such polymers are sold, for example, under the trade name Eastman AQ by the company Eastman Chemical Products.

The polymers of natural origin, modified or otherwise, can be selected from shellac, sandarac gum, dammars, elemis, copals, cellulosic polymers such as nitrocellulose, cellulose acetate, cellulose acetobutyrate, cellulose acetopropionate, ethyl cellulose, and mixtures thereof.

Advantageously, the first film-forming composition can comprise a styrene/acrylate copolymer dispersed in an aqueous medium or else nitrocellulose dissolved in an organic solvent medium, this organic solvent medium possibly comprising an organic solvent selected from ethyl acetate, butyl acetate and isopropyl alcohol; the second film-forming composition can comprise a styrene/acrylate copolymer dispersed in an aqueous medium.

The first and/or second film-forming polymers can be dissolved or dispersed in particle form in the corresponding cosmetically acceptable medium of each composition according to the invention. Respectively, the first and the second film-forming polymer can be present, in general, in an amount ranging from 1% to 70% by weight relative to the total weight, respectively, of the first and second composition, and preferably ranging from 10% to 40% by weight.

As the organic solvent which can be used in the invention, mention may be made of:

ketones which are liquid at room temperature, such as methyl ethyl ketone, methyl isobutyl ketone, diisobutyl ketone, isophorone, cyclohexanone and acetone;

alcohols which are liquid at room temperature, such as ethanol, isopropanol, diacetone alcohol, 2-butoxyethanol and cyclohexanol;

glycols which are liquid at room temperature, such as ethylene glycol, propylene glycol, pentylene glycol and glycerol;

propylene glycol ethers which are liquid at room temperature, such as propylene glycol monomethyl ether, propylene glycol monomethyl ether acetate, and dipropylene glycol mono-n-butyl ether;

short-chain esters (having from 3 to 8 carbon atoms in total), such as ethyl acetate, methyl acetate, propyl acetate, n-butyl acetate and isopentyl acetate;

ethers which are liquid at room temperature, such as diethyl ether, dimethyl ether or dichlorodiethyl ether;

alkanes which are liquid at room temperature, such as decane, heptane, dodecane and cyclohexane;

cyclic aromatic compounds which are liquid at room temperature, such as toluene and xylene;

aldehydes which are liquid at room temperature, such as benzaldehyde and acetaldehyde; and

mixtures thereof.

These solvents are especially suitable for nailcare and nail makeup: the composition in that case constitutes a nail varnish or a nailcare product.

In each composition having an organic solvent medium, the organic solvent can be present in an amount generally ranging from 30 to 99% by weight relative to the total weight of each composition, and more specifically from 60% to 90% by weight.

When the compositions for the implementation of the method according to the invention comprise an aqueous medium, the latter can comprise water. The amount of water in each composition having an aqueous medium can generally range from 30 to 99% by weight relative to the total weight of each composition, and more particularly from 60% to 90% by weight.

When the film-forming polymer does not make it possible to obtain, alone, a film having the characteristics mentioned above, it is possible to add a compound whose function is to modify the properties of the film-forming polymer so as to obtain the film having the desired hardness. In addition, in accordance with one embodiment of the method according to the invention, it is possible to add to the film-forming polymer at least one film-forming auxiliary which makes it possible to obtain a film having the characteristics as described above. The film-forming auxiliary makes it possible in particular to obtain a film having the desired hardness for the implementation of the method according to the invention.

A film-forming auxiliary of this kind can be selected from all of the compounds which are known to the person skilled in the art to be capable of fulfilling the desired function, and can in particular be selected from plasticizers.

Furthermore, when one of the compositions for the implementation of the method according to the invention comprises a film-forming polymer in the form of particles dispersed in the medium of the composition, the film-forming auxiliary can also be selected from coalescents.

In particular, mention may be made, alone or in a mixture, of the customary plasticizers or coalescants, such as:

glycols and their derivatives, such as diethylene glycol ethyl ether, diethylene glycol methyl ether, diethylene glycol butyl ether or else diethylene glycol hexyl ether, ethylene glycol ethyl ether, ethylene glycol butyl ether and ethylene glycol hexyl ether;

glycerol esters;

derivatives of propylene glycol, and especially propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,

esters of acids, especially carboxylic acids, such as citrates, phthalates, adipates, carbonates, tartrates, phosphates and sebacates;

ethoxylated derivatives, such as ethoxylated oils, especially vegetable oils such as castor oil; and silicone oils;

water-soluble polymers having a low glass transition temperature of less than 25° C., preferably less than 15° C.; and

mixtures thereof.

The amount of film-forming auxiliary can be selected by the person skilled in the art on the basis of his or her general knowledge so as to obtain a polymeric system which leads to a film having the desired mechanical properties while retaining cosmetically acceptable properties in the composition. For each composition, the amount of film-forming auxiliary can, for example, generally range from 0.5% to 20% by weight relative to the total weight of each composition, and more particularly from 2 to 10% by weight.

Each composition according to the invention may additionally comprise any additive known to the person skilled in the art as being capable of being incorporated into such a composition, such as thickeners, levelling agents, wetting agents, dispersants, antifoams, preservatives, UV filters, dyes, pigments, active substances, surfactants, moisturizers, perfumes, neutralizing agents, stabilizers and antioxidants. The person skilled in the art will of course take care to select this or these optional complementary compounds, and/or their amount, such that the advantageous properties of the composition according to the invention are not, or not substantially, impaired by the envisaged addition.

Each composition according to the invention can be prepared by the person skilled in the art on the basis of his or her general knowledge and in accordance with the state of the art.

The invention is illustrated in more detail in the following examples.

### EXAMPLE 1

a) A nail varnish having the composition 1 below is prepared in order to form the first layer 1:

| | | |
|---|---|---|
| aqueous dispersion of styrene/ | 18.8 | g of a.s. |
| acrylate copolymer | | |
| coalescant | 2.3 | g |
| plasticizer | 1.5 | g |
| ethyl alcohol | 5 | g |
| thickeners | 0.4 | g |
| preservatives | qs | |
| pigments | 1.2 | g |
| glycerine | 0.75 | g |
| water | qs to 100 | g |

The film obtained after drying has a hardness, measured with the aid of the Persoz pendulum, of between 20 and 25 seconds.

This composition is applied with the aid of a brush to the surface of unmade-up nails, in the form of a single layer.

b) A nail varnish having the composition 2 below is prepared in order to form the first layer 2:

| | | |
|---|---|---|
| aqueous dispersion of styrene/ | 19.7 | g of a.s. |
| acrylate copolymer | | |
| coalescant | 2.7 | g |
| ethyl alcohol | 5 | g |
| thickeners | 0.5 | g |
| preservatives | qs | |
| pigments | 3.3 | g |
| glycerine | 1.8 | g |
| water | qs to 100 | g |

The film obtained after drying has a hardness, measured with the aid of the Persoz pendulum, of between 60 and 65 seconds.

This composition 2 is applied with the aid of a brush to the entirety of the dry layer 1. After drying, the layer 2 is crackled over the entirety of its surface, in a regular manner, and the crackle-surface makeup exhibits a good hold, in particular with respect to rubbing with the fingers. The result is therefore a nail makeup having a good hold and featuring an original, fissured, crackle effect.

The cracks can also be obtained by accelerating the drying of the layer 2 by means of a heat source such as a hairdryer or a source of pulsed air such as a fan.

### EXAMPLE 2: COMPARATIVE

The composition 2 of Example 1 has been applied directly to unmade-up nails. After drying, the film cracks on the nail but the cracks are not resistant to rubbing with the finger. The crackle film does not hold to the nail, and breaks up.

### EXAMPLE 3

a) The composition 1 of Example 1 is applied using a brush to the surface of unmade-up nails, in the form of a single layer.

b) A nail varnish having the following composition 2 is prepared in order to form the first layer 2:

| | | |
|---|---|---|
| nitrocellulose | 11 | g |
| plasticizers | 5.7 | g |
| thickeners | 1.3 | g |
| preservatives | qs | |
| pigments | 1.2 | g |
| solvents (ethyl acetate, butyl | qs to 100 | g |
| acetate, isopropyl alcohol) | | |

The film obtained after drying has a hardness, measured with the aid of the Persoz pendulum, of approximately 70 seconds.

This composition 2 is applied with the aid of a brush to the entirety of the dry layer 1. After drying, the layer 2 is crackled over the entirety of its surface, in a regular manner, and the crackle-surface makeup exhibits a good hold, in particular with respect to rubbing with the fingers.

What is claimed is:

1. A method of making up skin, lips or an exoskeletal appendage, comprising:
applying to said skin, lips or exoskeletal appendage a first layer of a first film-forming composition comprising a cosmetically acceptable medium and at least one first film-forming polymer, said first composition being capable of forming a first film having a hardness d1 ranging from 20 to 85 seconds, and
then applying to at least one part of the first layer a second layer of a second film-forming composition comprising a cosmetically acceptable medium and at least one second film-forming polymer, said second composition being capable of forming a second film having a hardness d2 such that the difference between d2 and d1 is greater than or equal to 10 seconds.

2. The method according to claim 1, wherein said exoskeletal appendage is chosen from nails, eyelashes, eyebrows and hair.

3. The method according to claim 1, wherein said difference between d2 and d1 ranges from 10 to 360 seconds.

4. The method according to claim 1, wherein said difference between d2 and d1 ranges from 10 to 120 seconds.

5. The method according to claim 1, wherein said hardness d1 ranges from 20 to 40 seconds.

6. The method according to claim 1, wherein said hardness d2 ranges from 30 to 180 seconds.

7. The method according to claim 1, wherein said hardness d2 ranges from 80 to 120 seconds.

8. The method according to claim 1, wherein at least one of said first film-forming composition and said second film-forming composition comprises an organic solvent medium.

9. The method according to claim 1, wherein at least one of said first film-forming composition and said second film-forming composition comprises an aqueous medium.

10. The method according to claim 1, wherein at least one of said at least one first film-forming polymer and said at least one second film-forming polymer is dissolved in the corresponding cosmetically acceptable medium.

11. The method according to claim 1, wherein at least one of said at least one first film-forming polymer and said at least one second film-forming polymer is dispersed in particle form in the corresponding cosmetically acceptable medium.

12. The method according to claim 1, wherein said at least one first film-forming polymer and said at least one second film-forming polymers are chosen independently of one another from free-radical polymers, polycondensates and polymers of natural origin.

13. The method according to claim 1, wherein said at least one first film-forming polymer and said at least one second film-forming polymers are chosen independently of one another from vinyl polymers, polyurethanes, polyesters and cellulosic polymers.

14. The method according to claim 1, wherein said at least one first film-forming polymer and said at least one second film-forming polymer are present in an amount ranging from 1% to 70% by weight relative to the total weight, respectively, of said first and said second composition.

15. The method according to claim 1, wherein said at least one first film-forming polymer and said at least one second film-forming polymer are present in an amount ranging from 10% to 40% by weight relative to the total weight, respectively, of said first and said second composition.

16. The method according to claim 1, wherein at least one of said first film-forming composition and said second film-forming composition comprises at least one film-forming auxiliary.

17. The method according to claim 16, wherein said at least one film-forming auxiliary is present in an amount ranging from 0.5% to 20% by weight relative to the total weight of at least one of said first film-forming composition and said second film-forming composition.

18. The method according to claim 17, wherein said at least one film-forming auxiliary is present in an amount ranging from 2 to 10% by weight relative to the total weight of at least one of said first film-forming composition and said second film-forming composition.

19. The method according to claim 1, wherein at least one of said first film-forming composition and said second film-forming composition comprises at least one plasticizer.

20. The method according to claim 1, wherein at least one of said first film-forming composition and said second film-forming composition comprises at least one coalescent.

21. The method according to claim 1, wherein at least one of said first film-forming composition and said second film-forming composition comprises at least one additive chosen from thickeners, levelling agents, wetting agents, dispersants, antifoams, preservatives, UV filters, dyes, pigments, active substances, surfactants, moisturizers, perfumes, neutralizing agents, stabilizers and antioxidants.

22. The method according to claim 1, wherein at least one of said first film-forming composition and said second film-forming composition is in the form chosen from a nail varnish, a rouge, an eyeshadow, a foundation, a lip and a body makeup product.

23. The method according to claim 1, wherein said second layer is dried under acceleration with the aid of at least one drying aid chosen from a source of heat and a source of pulsed air.

24. The method according to claim 1, wherein said at least one first film-forming composition comprises a styrene/acrylate copolymer dispersed in an aqueous medium.

25. The method according to claim 1, wherein said at least one first film-forming composition comprises a nitrocellulose dissolved in an organic solvent medium.

26. The method according to claim 1, wherein said at least one second film-forming composition comprises a styrene/acrylate copolymer dispersed in an aqueous medium.

27. A makeup product for skin, lips, or an exoskeletal appendage obtained by:
applying to said skin, lips or exoskeletal appendage a first layer of a first film-forming composition comprising a cosmetically acceptable medium and at least one first film-forming polymer, said first composition being capable of forming a first film having a hardness d1 ranging from 20 to 85 seconds, and
then applying to at least one part of the first layer a second layer of a second film-forming composition comprising a cosmetically acceptable medium and at least one second film-forming polymer, said second composition being capable of forming a second film having a hardness d2 such that the difference between d2 and d1 is greater than or equal to 10 seconds.

28. A makeup kit comprising at least two compartments, a first compartment comprising a first film-forming composition and a second compartment comprising a second film-forming composition wherein:
said first film-forming composition comprises a cosmetically acceptable medium and at least one first film-forming polymer, said first composition being capable of forming a first film having a hardness d1 ranging from 20 to 85 seconds; and
said second film-forming composition comprises a cosmetically acceptable medium and at least one second film-forming polymer, said second composition being capable of forming a second film having a hardness d2 such that the difference between d2 and d1 is greater than or equal to 10 seconds.

29. The makeup kit according to claim 28, wherein said difference between d2 and d1 ranges from 10 to 360 seconds.

30. The makeup kit according to claim 28, wherein said difference between d2 and d1 ranges from 10 to 120 seconds.

31. The makeup kit according to claim 28, wherein said hardness d1 ranges from 20 to 40 seconds.

32. The makeup kit according to claim 28, wherein said hardness d2 ranges from 30 to 180 seconds.

33. The makeup kit according to claim 28, wherein said hardness d2 ranges from 80 to 120 seconds.

34. The makeup kit according to claim 28, wherein at least one of said first film-forming composition and said second film-forming composition comprises an organic solvent medium.

35. The makeup kit according to claim 28, wherein at least one of said first film-forming composition and said second film-forming composition comprises an aqueous medium.

36. The makeup kit according to claim 28, wherein at least one of said at least one first film-forming polymer and said at least one second film-forming polymer is dissolved in the corresponding cosmetically acceptable medium.

37. The makeup kit according to claim 28, wherein at least one of said at least one first film-forming polymer and said at least one second film-forming polymer is dispersed in particle form in the corresponding cosmetically acceptable medium.

38. The makeup kit according to claim 28, wherein said at least one first film-forming polymer and said at least one second film-forming polymer are chosen independently of one another from free-radical polymers, polycondensates and polymers of natural origin.

39. The makeup kit according to claim 28, wherein said at least one first film-forming polymer and said at least one second film-forming polymer are chosen independently of one another from vinyl polymers, polyurethanes, polyesters and cellulosic polymers.

40. The makeup kit according to claim 28, wherein said at least one first film-forming polymer and said at least one second film-forming polymer are present in an amount ranging from 1% to 70% by weight relative to the total weight, respectively, of said first and said second composition.

41. The makeup kit according to claim 28, wherein said at least one first film-forming polymer and said at least one second film-forming polymer are present in an amount ranging from 10% to 40% by weight relative to the total weight, respectively, of said first and said second composition.

42. The makeup kit according to claim 28, wherein at least one of said first film-forming composition and said second film-forming composition comprises at least one film-forming auxiliary.

43. The makeup kit according to claim 42, wherein said at least one film-forming auxiliary is present in an amount ranging from 0.5% to 20% by weight relative to the total weight of at least one of said first and said second composition.

44. The makeup kit according to claim 42, wherein said at least one film-forming auxiliary is present in an amount ranging from 2 to 10% by weight relative to the total weight of at least one of said first and said second composition.

45. The makeup kit according to claim 28, wherein at least one of said first film-forming composition and said second film-forming composition comprises at least one plasticizer.

46. The makeup kit according to claim 28, wherein at least one of said first film-forming composition and said second film-forming composition comprises at least one coalescent.

47. The makeup kit according to claim 28, wherein at least one of said first film-forming composition and said second film-forming composition comprises at least one additive chosen from thickeners, levelling agents, wetting agents, dispersants, antifoams, preservatives, UV filters, dyes, pigments, active substances, surfactants, moisturizers, perfumes, neutralizing agents, stabilizers and antioxidants.

48. The makeup kit according to claim 28, wherein at least one of said first film-forming composition and said second film-forming composition is in the form chosen from a nail varnish, a rouge, an eyeshadow, a foundation, a lip and a body makeup product.

49. The makeup kit according to claim 28, wherein said at least one first film-forming composition comprises a styrene/acrylate copolymer dispersed in an aqueous medium.

50. The makeup kit according to claim 28, wherein said at least one first film-forming composition comprises a nitrocellulose dissolved in an organic solvent medium.

51. The makeup kit according to claim 28, wherein said at least one second film-forming composition comprises a styrene/acrylate copolymer dispersed in an aqueous medium.

52. The makeup kit according to claim 28, wherein said kit comprises means for applying said first film-forming composition and said second film-forming composition to skin, lips, or an exoskeletal appendage.

53. The makeup kit according to claim 52, wherein said means are chosen from coarse and fine brushes, pens and sponges.

54. A made-up substrate chosen from skin, lips or an exoskeletal appendage, wherein said substrate comprises a first layer of a first film-forming composition comprising a cosmetically acceptable medium and at least one first film-forming polymer, said first composition being capable of forming a first film having a hardness d1 ranging from 20 to 85 seconds, and applied to at least one part of the first layer a second layer of a second film-forming composition comprising a cosmetically acceptable medium and at least one second film-forming polymer, said second composition being capable of forming a second film having a hardness d2 such that the difference between d2 and d1 is greater than or equal to 10 seconds.

55. The made up substrate according to claim 54, wherein said substrate is in a form chosen from false nails, false eyelashes and hairpieces.

56. A method of obtaining a crackle-surface makeup having good hold comprising applying to a substrate chosen from skin, lips or an exoskeletal appendage a first layer of a first film-forming composition comprising a cosmetically acceptable medium and at least one first film-forming polymer, said first composition being capable of forming a first film having a hardness d1 ranging from 20 to 85 seconds, and then applying to at least one part of the first layer a second layer of a second film-forming composition comprising a cosmetically acceptable medium and at least one second film-forming polymer, said second composition being capable of forming a second film having a hardness d2 such that the difference between d2 and d1 is greater than or equal to 10 seconds.

57. The method of claim 56, wherein said first film-forming composition comprises a styrene/acrylate copolymer dispersed in an aqueous medium or nitrocellulose dissolved in an organic solvent medium and further wherein said second film-forming composition comprises styrene/acrylate copolymer dispersed in an aqueous medium.

58. A method of obtaining a crackle-surface makeup having good hold comprising applying to a substrate chosen from skin, lips or an exoskeletal appendage a film-forming composition comprising a cosmetically acceptable medium and at least one first film-forming polymer, said first composition being capable of forming a film having a hardness d1 ranging from 20 to 85 seconds.

59. The method of claim 58, wherein said film-forming composition comprises a styrene/acrylate copolymer dispersed in an aqueous medium or nitrocellulose dissolved in an organic solvent medium.

60. A method of obtaining a crackle-surface makeup having good hold on a substrate chosen from skin, lips or an exoskeletal appendage comprising applying to all or part of a first film layer having a hardness d1 ranging from 20 to 85 seconds on said substrate a second layer of a second film-forming composition comprising a cosmetically acceptable medium and at least one second film-forming polymer, said second composition being capable of forming a second film having a hardness d2 such that the difference between d2 and d1 is greater than or equal to 10 seconds.

61. The method of claim 60, wherein said film-forming composition comprises a styrene/acrylate copolymer dispersed in an aqueous medium.
